(19) United States
(12) Patent Application Publication
(10) Pub. No.: US 2012/0239032 A1
(43) Pub. Date: Sep. 20, 2012

(54) MICRO-STEERABLE CATHETER

(75) Inventors: Qiming Zhang, State College, PA (US); Shihai Zhang, State College, PA (US); Brian Zellers, State College, PA (US)

(73) Assignee: Strategic Polymer Sciences, Inc., State College, PA (US)

(21) Appl. No.: 13/482,901

(22) Filed: May 29, 2012

Related U.S. Application Data
(62) Division of application No. 11/898,475, filed on Sep. 12, 2007.
(60) Provisional application No. 60/951,133, filed on Jul. 20, 2007.

(51) Int. Cl. A61B 18/18

(52) U.S. Cl. 606/41

(57) ABSTRACT

Micro-streerable catheters for use in delivering therapeutic treatment in the body, such as ablation and cauterization, and which exhibit precise movement are disclosed. Embodiments include electrical micro-catheters that comprise of electroactive polymers. A preferred embodiment includes a programmable catheter.

This application claims the benefit of U.S. Provisional Application 60/951,133 which was filed on Jul. 20, 2007, said application being incorporated by reference in its entirety.

### BACKGROUND OF THE DISCLOSURE

1. Field of the Disclosure

The present disclosure relates to electrical micro-steerable catheters, which use electroactive polymers to provide micro-articulation. The present disclosure also relates to utilizing electrical micro-steerable catheters for programmable micro-steerable catheters.

2. Background

Medical procedures like cardiac ablation, cardio-vascular diagnostics, angioplasty, and stenting rely on catheter technology. This also pertains to other surgical procedures involving the brain, the gastro-intestinal tract, and the urethra.

As an example, an ablation catheter is used to treat both ventricular and supraventricular tachycardia. To achieve the surgical goal, the catheter is first sent to the desired location or locations inside the patient body through a specific pathway. The catheter then serves as a protective path through which destructive energy can be directed to the abnormal area and ablate the tissue. The ablation energy sources include radiofrequency electrical energy, laser energy, direct current energy, microwave energy, cryogenic energy, etc. However, a major drawback of the ablation catheters currently in use is their inability to effectively steer the catheter tip to the desired locations in the body.

An example of a current ablation catheter is shown in FIG. 1, where the catheter has a guide wire lumen. The physician steers the tip of the catheter by manipulating (pulling, pushing, twisting, etc.) the guide wires (see e.g., U.S. Pat. No. 6,183,435). As a result, these catheters are difficult to steer and control, which is primarily why an ablation procedure takes about 6 to 8 hours.

As such, current ablation procedures are tedious and costly. This adds to patient risk and limits the number of patients who receive these procedures. The length of the procedure and associated costs cause many prospective cardiac ablation candidates to forgo catheter procedures.

Further, coronary diagnostic procedures such as angioplasty and stenting could be improved by more precise devices. Most of these procedures use at least 3 or 4 catheters because the catheter device tips have to be changed and bent in order to reach various locations in the coronary arteries. Similar to the ablation catheter, the diagnostic catheter's shortcomings add risk, time, and cost to the overall procedure. In some cases, patients will remain untreated because the catheter could not reach the desired location. In other cases, the procedure may take 3 to 4 hours. The cardiologist must extract the catheter from the patient several times in order to change tip sizes to reach different areas. Consequently, the hospital has to stock a much larger inventory of catheters because it is not known which catheter will be used. This repeated catheter extraction and insertion may also increase risk of infections and trauma to the patient.

Several recent disclosures have been aimed at improving catheter steerability. One recent steering method teaches the use of pressurized fluid. The catheter consists of one or more lumens for holding fluid. By applying pressure, the pressurized lumen will bend in a specific direction (see, e.g., US 2007/0060997).

Another approach utilizes robotic devices to steer the catheter to desired locations (see, e.g., U.S. Pat. No. 6,770,081 B1, US 2006/0293643 A1, 2007/0043338 A1). These catheters require complex mechanical and electrical mechanisms and the articulations are realized through a remote computer. Haptic tactile feedback mechanisms can also be incorporated to prevent damage of the tissue.

Yet another type of catheter uses actuators made from magnetostrictive materials. The actuators respond to the magnetic field outside the patient and generate desirable actuations. Since large magnets are required to generate the required magnetic field, the complete system occupies a large room and is expensive.

Furthermore, traditional Electroactive Polymers, (EAPs), such as piezoelectric polyvinylidene fluoride (PVDF) polymers that are used in soft actuators, suffer from low electrically actuated strain (about 0.1%). Consequently, these EAPs do not generate the large motions that are preferable for electrical micro-steerable catheters.

Therefore, there currently exists a need for micro-steerable catheters, which are more effective and practical.

### SUMMARY OF THE DISCLOSURE

The present disclosure relates to a micro-steerable catheter and methods of using the micro-steerable catheter. An advantageous feature of the micro-steerable catheter described in this disclosure is that its movement (micro-articulation) can be made in a precise manner under control of electric voltages. This is especially advantageous in any minimally invasive surgical procedure used to repair vessels in the heart, brain, urethra, or other organ. Furthermore, the catheter's movement can also be computer controlled which enables programmability.

An additional advantage of the micro-steerable catheter of this disclosure is that it can replace the majority of catheters hospitals have to keep in stock. The reason for this is that the feature of micro-steerability minimizes the need to change tips and to bend a multitude of catheters used in a procedure in order to reach a precise location. This also reduces patient risk and contributes to the overall quality of the procedure.

One aspect of the catheters described herein is the use of an electroactive polymer to steer the catheter. In one embodiment, an electrical, microsteerable catheter can be made by simply replacing the plastic sheath in existing catheters with an electroactive polymer. Further, the electroactive polymer can be patterned with separate electrode segments and sections within segments, which can be individually controlled through a remote computer.

Another aspect of the catheters described herein is a method for steering a catheter to different positions by varying voltage levels. By such a method the catheters having an electroactive polymer can be steered and controlled to various desired locations during an operation with a high degree of precision (e.g., a precision to within about 0.1 mm), which exceeds catheter technologies without the EAP.

A further aspect of the disclosure is the use of electrostrictive poly(vinylidene fluoride) based polymers, including terpolymers of electrostrictive poly(vinylidene fluoride-trifluoroethylene-cholorofluoroethylene) and other terpolymers with similar electromechanical performance (see, e.g., U.S. Pat. No. 6,787,238), and a high energy particle irradiated P(VDF-TrFE) and other related irradiated PVDF based polymers (see, e.g., U.S. Pat. No. 6,423,412), which can generate large strain (more than 4% strain with an elastic modulus higher than 0.5 GPa) under an electric field.

Another aspect of the disclosure is a combination of an EAP with a shape memory polymer (SMP). Here, shapes resulting from bending actuation may be maintained by the memory effect of the SMP in the absence of the applied electric field responsible for the actuation.

Another aspect of the disclosure is the programmable electrical micro-steerable catheter. The electrical micro-steerable catheter design lends itself to programmability as the catheter tip position can be precisely controlled by voltage signals applied to the electroactive polymer sheath, which are well suited to computer control. Computer control can be made to vary the applied voltages to different electroactive polymer segments, and sections within a segment, to induce a desired steerable catheter shape, such that the catheter tip can reach a target location in the body. For instance, the values of the applied voltages necessary to achieve a desired catheter shape for the tip to reach a predetermined target in the patient body can be directly inputted into the catheter controller. The voltage value is stored until the desired shape is called upon by the user. This procedure can be repeated for all the target positions in the treatment and hence a computer may control the whole operation, with physicians monitoring the whole treatment process.

### BRIEF DESCRIPTION OF THE DRAWINGS

FIG. 1 shows the articulating portion of a conventional commercially available ablation catheter.

FIG. 2a shows the simulation results for the electrical micro-steerable catheter with electroactive polymers.

FIG. 2b shows the simulation results for the electrical micro-steerable catheter with electroactive polymers (same as that in FIG. 2a). The simulation shows both the deflection and the blocked force in grams, resulting from the application of an electric field.

FIG. 3 shows the electromechanical response of a P(VDF-TrFE-CFE) terpolymer, S₃ is the thickness strain and S₁ is along the film surface and in the film stretched direction.

FIG. 4 shows bending of a prototype EAP unimorph actuator.

FIG. 5 shows the cross section of a hybrid actuator consisting of a SMP layer 20 bonded to an active (electrode) EAP layer 10. The hybrid actuator can have many different kinds of combination of EAP and SMP. For example, a hybrid actuator can have an EAP layer bonded between two SMP layers, or one EAP layer and one SMP layer bonded together.

FIG. 6 shows the use of a SMP in the intended application.

FIG. 7 shows an orthographic view of the entire length of an electrical micro-steerable catheter according to another embodiment of the disclosure.

FIG. 8 shows the cross section of the steerable catheter of FIG. 7 with 4 active EAP sections around the circumference.

FIG. 9 shows the layering of an electroactive polymer groups within a segment according to an embodiment of the disclosure.

FIG. 10 shows a portion of a continuous sheet of electroactive polymer with discrete areas of conducting polymer (or other electrode) deposited on its surface, according to another embodiment of the disclosure.

FIG. 11 shows how each electroactive polymer section in a segment is electrically connected to its separate driving voltage for the electrode pattern in FIG. 9.

FIG. 12 shows an orthographic view of one electroactive polymer section from one electroactive polymer segment.

FIG. 13 is an illustration of the catheter tip and an ablation target location.

FIG. 14 shows a simulation of the optimization procedure for an electrical micro-steerable catheter tip to reach the ablation target with a pre-determined precision.

FIG. 15 is another simulation of the optimization procedure for an electrical micro-steerable catheter tip to reach an ablation target with a pre-determined precision.

FIG. 16a shows a continuous line representing the ablation target position.

FIG. 16b illustrates a discretization of the target line into a series of discrete target points,

FIG. 17 illustrates the translational degree of freedom of the catheter base.

### DETAILED DESCRIPTION OF THE DISCLOSURE

The following detailed description is exemplary in nature and is not intended to limit the scope, applicability, or configuration of the disclosure in any way. Rather, the following description provides a practical illustration for implementing exemplary embodiments of the disclosure.

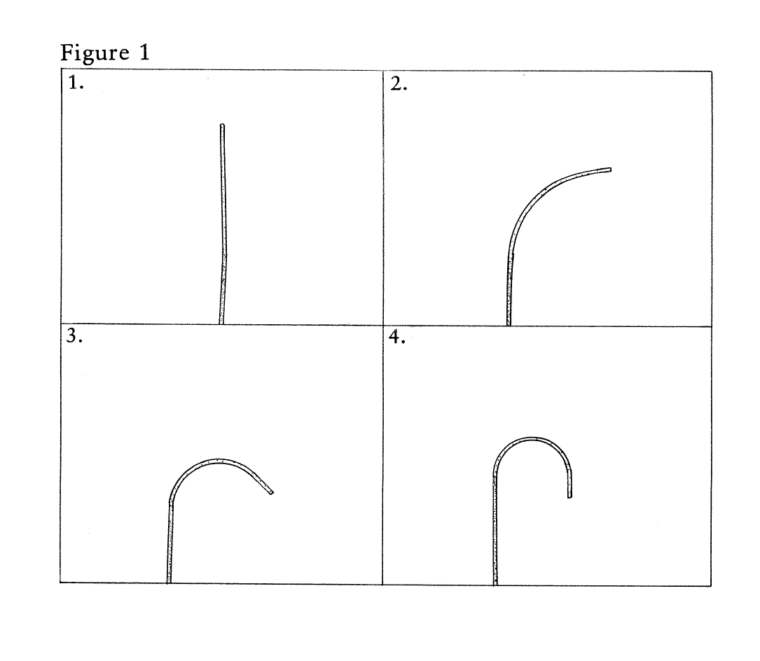

The present disclosure relates to a micro-steerable catheter comprising an electroactive polymer (EAP) for steering the catheter and method of using such a catheter. For comparison, FIG. 1 shows the articulating portion of a conventional commercially available ablation catheter. The catheter can only bend in one direction with the pull wire (See, e.g., U.S. Pat. No. 6,979,312). In order for the catheter tip to reach all positions in the treatment volume, the physician has to manually twist (rotate) the catheter by hand in addition to the bending achieved by the pull wire. The bending induced by the pull wire is not precise and not reproducible. The combination of the imprecision in the twist of the catheter from a proximal location and the bending by using pull wires makes it difficult to direct the catheter tip to a location of concern inside the body. The articulating length of the catheter is 7 cm long. The starting position is labeled 1, and reflects the shape of the articulating portion with no user input. Positions 2 through positions 4 show the progression of the articulation as the user input is increased. Although the catheter can be steered and its shape can be changed, its control mechanism does not permit the catheter tip to be moved to a predetermined position. Moreover, the bending of the catheter can only be in one direction.

In contrast, the catheters of the present disclosure include an electroactive polymer (EAP). An advantageous feature of the catheters described herein is that they can be made simply and at a low cost. For example, an electrical, micro-steerable catheter can be made by simply replacing the plastic sheath of a conventional catheter, such as shown in FIG. 1, with an electroactive polymer or by replacing the plastic sheath of a conventional catheter with an EAP patterned with electrode segments, which can be individually controlled through a remote computer. As used herein a sheath includes a layer over, in whole or in part, an inner lumen. A catheter comprising an EAP can be steered to different positions by applying different voltage levels. The electrical micro-steerable catheter can allow bending in all the directions, which is in contrast with conventional commercial catheters which can bend in only one direction. The one-to-one correspondence of the bending direction and the degree of bending of the electrical micro-steerable catheters enables the catheter tip to be moved to a predetermined position with very high precision (within about 0.1 mm).

The micro-steerable catheters of the present disclosure may be used, for example, in cardiac ablation and cardio-diagnostic procedures. The micro-steerable catheters of the present disclosure may also be used in the brain and urethra tract. The electrical steerable catheter in this disclosure can reduce the time of cardiac ablation procedures to about 2-3 hours or approximately ⅓ the time that the procedure currently takes. Reduced surgical time will: 1) decrease patient risk for complications, 2) allow more patients to be treated, 3) reduce the cost of the procedure to patients and healthcare providers, and 4) allow more physicians to practice electrophysiology procedures.

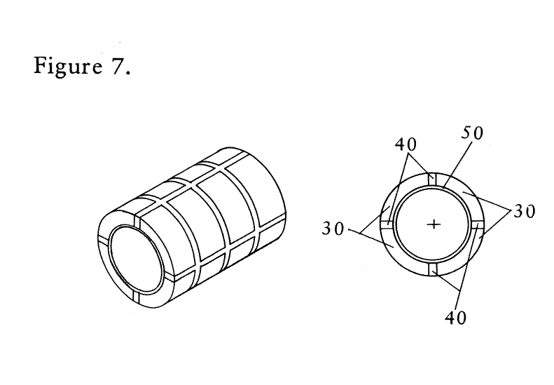

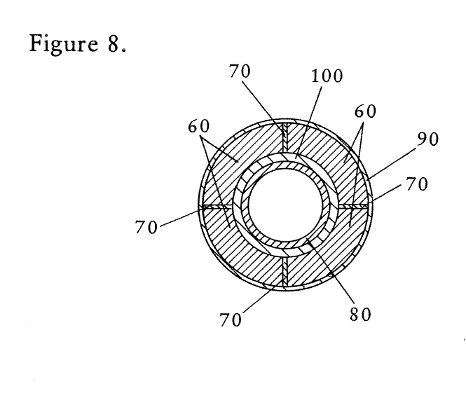

In a preferred embodiment, the steerable portion of catheter 13 in the form of a sheath and made of a sheet of electroactive polymer (EAP), which is preferably rolled into a multilayer tube. The EAP tube can be patterned with segmented electrodes as illustrated in FIG. 7. Along the catheter length direction, the electrode segment can have a single segment, or more than one segment, depending on the intended usage. Each segment can contain a total of n individual sections (FIG. 8 illustrates an electrical micro-steerable catheter with four sections), where n can be 2 or any number greater than 2.

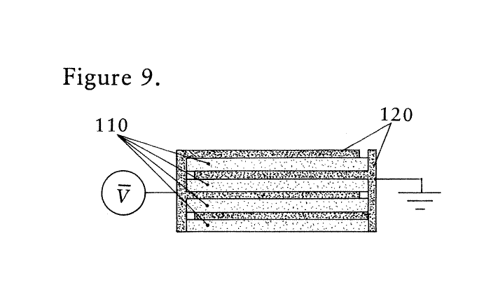

The EAP portion of the catheter can comprise multiple layers of EAP where each layer has an electrically conductive electrode on its outer surface, referred to as an active EAP (FIG. 9). The active EAP sections can be positioned about the catheter circumference (FIG. 8). Each active EAP section within a segment is typically electrically isolated from other active EAP sections in the same segment as well as those in adjacent segments. This isolation can be achieved through EAP material that does not have a conductive electrode, referred to as an inactive EAP.

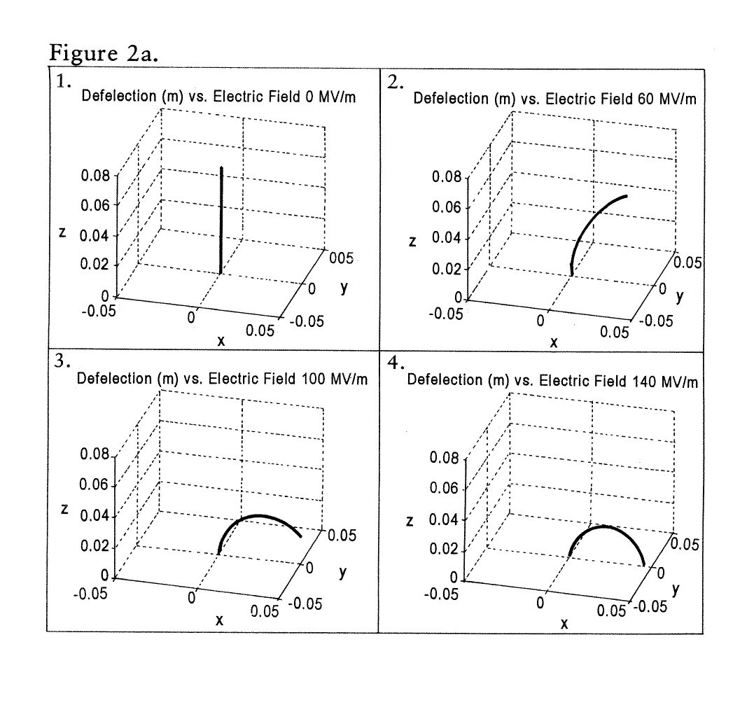

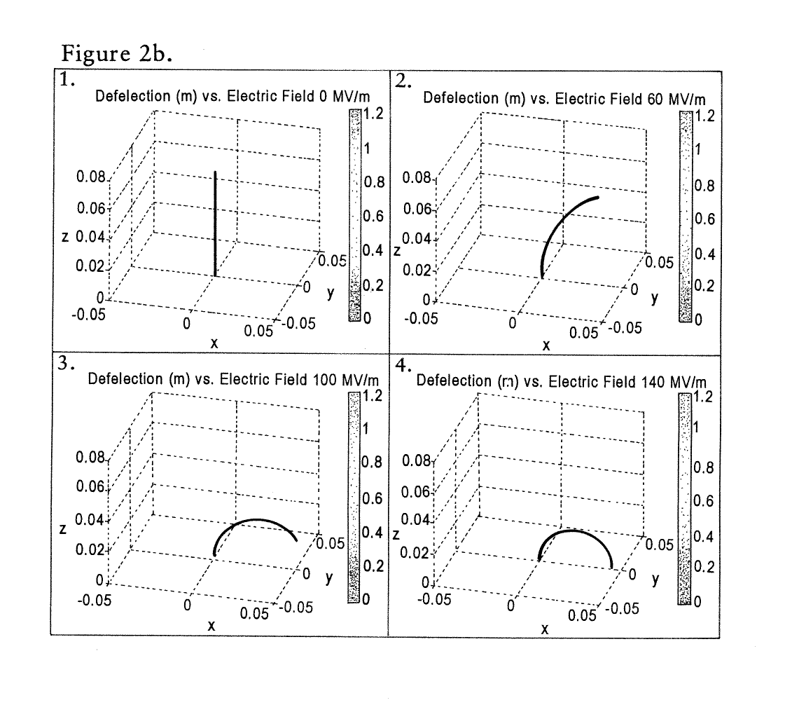

FIG. 2a shows the simulation results for an electrical micro-steerable catheter comprised of electroactive polymers. The simulation shows the articulation that is possible when an electric field is applied to the electroactive polymers. The simulation is based on the superposition of deflections achieved for electroactive polymer sections along the steerable catheter's length.

The activated sections are modeled as applied moments resulting from the extension of the energized polymer cross section (along the catheters axis) at a nonzero eccentricity from the neutral axis of the steerable catheter. Position 1 shows the steerable catheter with an applied electric field of 0 MV/m. Position 2 shows the steerable catheter with an applied electric field of 60 MV/m. Position 3 shows the steerable catheter with an applied electric field of 100 MV/m. Position 4 shows the steerable catheter with an applied electric field of 140 MV/m.

FIG. 2b shows the simulation results for an electrical micro-steerable catheter with electroactive polymers. The simulation shows both the deflection and the blocked force, in grams, resulting from the application of an electric field. The deflection is the same as that described in the discussion of FIG. 2a. The blocked force is the force required to prevent the deflection of the steerable catheter. Position 1 shows both the deflection and blocked force resulting from an electric field of 0 MV/m. Position 2 shows both the deflection and blocked force resulting from an electric field of 60 MV/m. Position 3 shows both the deflection and blocked force resulting from an electric field of 100 MV/m. Position 4 shows both the deflection and blocked force resulting from an electric field of 140 MV/m.

In another embodiment, the catheter can include an electroactive polymer comprising a high energy electron irradiated polymer, such as irradiated P(VDF-TrFE) and other related polymers. These EAPs are described in U.S. Pat. No. 6,423,412 and are incorporated herein in their entirety by reference.

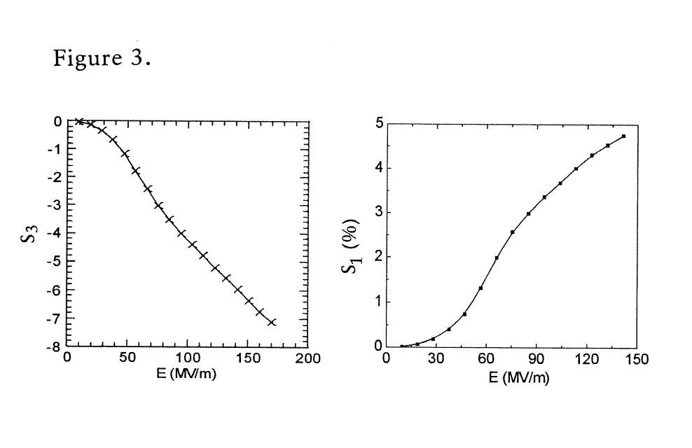

In a further embodiment of the disclosure, the actuator comprises a P(VDF-TrFE-CFE) terpolymer. The electromechanical response of a P(VDF-TrFE-CFE) terpolymer is shown in FIG. 3. S₃ is the strain of the terpolymer in the thickness direction; a negative S₃ indicates the terpolymer layer becomes thinner under an electric field. A thickness strain above 7% can be achieved under an applied electric field. S₁ is the strain in the transverse direction and the film sample becomes longer if an electric field is applied across the thickness direction. A transverse strain of 4.7% can be achieved for the terpolymer under 140 MV/m.

As illustrated in FIG. 3, a P(VDF-TrFE-CFE) electrostrictive polymer can generate a thickness strain of 7%, and a transverse strain of 5% (under film stretching direction). FIG. 3 shows the electromechanical response of a P(VDF-TrFE-CFE) terpolymer. S₃ is the thickness strain and S₁ is along the film surface and in the film stretched direction. Additional electrostrictive polymers that can be used with the present catheter, include: P(VDF-TrFE-CTFE), (CTFE: chlorotrifluoroethylene), Poly(vinylidene fluoride-trifluoroethylene-vinylidene chloride), poly(vinylidene fluoride-tetrafluoroethylene-chlorotrifluoroethylene), poly(vinylidene fluoride-trifluoroethylene-hexafluoropropylene), poly(vinylidene fluoride-tetrafluoroethylene-hexafluoropropylene), poly(vinylidene fluoride-trifluoroethylene-tetrafluoroethylene), poly(vinylidene fluoride-tetrafluoroethylene-tetrafluoroethylene), poly(vinylidene fluoride-tri fluoroethylene-vinyl fluoride), poly(vinylidene fluoride-tetrafluoroethylene-vinyl fluoride), poly(vinylidene fluoride-trifluoroethylene-perfluoro(methyl vinyl ether)), poly(vinylidene fluoride-tetrafluoroethylene-perfluoro (methyl vinyl ether)), poly(vinylidene fluoride-trifluoroethylene-bromotrifluoroethylene, polyvinylidene), poly(vinylidene fluoride-tetrafluoroethylene-chlorofluoroethylene), poly(vinylidene fluoride-trifluoroethylene-vinylidene chloride), and poly(vinylidene fluoride-tetrafluoroethylene vinylidene chloride). Furthermore, these EAPs possess a high elastic modulus, from 0.5 GPa in the uniaxially stretched P(VDR-TrFE-CFE) terpolymers to more than 1 GPa in the high energy electron irradiated P(VDF-TrFE) copolymers. The combination of high elastic modulus and high strain results in a high elastic energy density, making these EAPs advantageuos in providing large motions with high precision in the electrical micro-steerable catheters. Additional EAPs that can be used with the present catheter include those disclosed in U.S. Pat. No. 6,787,238 which are incorporated herein in their entirety.

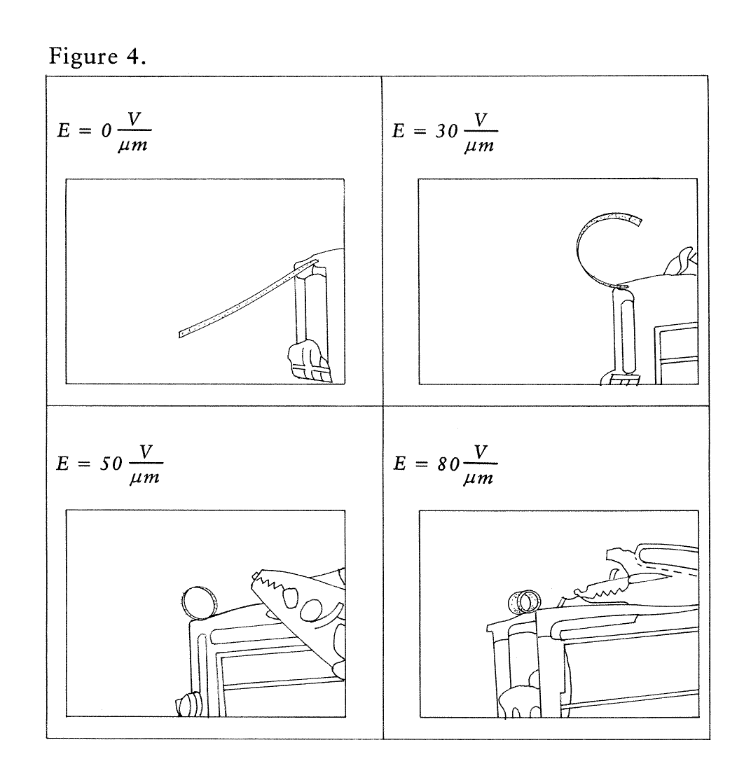

FIG. 4 illustrates the bending motion of an EAP actuator, which comprises two thin films of P(VDF-TrFE-CFE) polymer bonded together. When an electric field is applied to one terpolymer film, which becomes longer, a bending motion is created as can be seen in the figure. The degree of bending can be precisely controlled by the applied voltage (electric field E=voltage/film thickness). In the figure, the bending under E=30 V/μm, 50 V/μm, and 80 V/μm is shown. In FIG. 4, different actuation states correspond to different applied electric fields. In order to keep the actuator in a given bending position, an electric field is maintained to the EAP film.

The micro-articulation in FIG. 2a is from a simulated result using the strain data of the EAP in FIG. 3 with a modulus of 0.5 GPa (OD=2 mm, ID=0.7 mm, L=7 cm). For the results in FIG. 2a, the EAP has one segment along the catheter length and four sections about the circumference, and the micro-articulation is achieved by actuating two neighboring sections. By actuating (applying a voltage) on either a single section or combining two neighboring sections together, the catheter can be steered to different directions and any positions in 3-dimensional space with a precision better than 0.1 mm.

In another embodiment of the disclosure, a shape memory polymer, (SMP) is included in the sheath of the catheter steerable section. This is illustrated in FIG. 8. The normal temperature of the catheter is 37° C., the temperature of the human body. To steer the catheter tip to a desired position while making use of the SMP memory effect, the EAP and SMP section of the catheter is heated to T₀ (T₀ is in the range of 40 to 45° C., if allowed by body tissues), which is just above Tₜᵣₐₙₛ of the SMP but below the temperature which will cause damage to the tissue, so that the SMP is sufficiently soft to allow for reshaping. By applying a voltage to the EAP sections to steer the catheter tip to the predetermined position, the SMP is also reshaped. The total time for the temperature rising to T₀ should be less than about one minute or a time allowed by body tissue. The temperature of the EAP and SMP then returns to the temperature of the body (approximately 37° C.) and the SMP becomes stiff (possessing a high elastic modulus) and also memorizes the shape change. After that, the applied voltage on the EAP is removed and the catheter can be maintained in the articulated position without the need for an applied voltage to maintain the shape.

For the embodiments of the disclosure shown in FIG. 7 and FIG. 8, the inner diameter can be the same as that in the current conventional ablation catheters, which allows an ablation conductor to connect the ablation transducer and its corresponding driving signal generator.

In yet another embodiment, the EAP section is directly bonded to the circumference of an existing catheter, either inside or outside, whose length L is about 7 cm to about 10 cm. FIG. 8 also shows the arrangement of the total n active EAP sections about the cross section of the tube circumference. Along the tube length, the EAP can be separated into different segments, as illustrated in FIG. 7. The existing polymer sheath serves as the base structure of the catheter. The inner diameter of the tube allows the conductors to connect the active EAP segments, as well their sections, and ablation transducers to their respective driving signal generators. The outer diameter of the active EAP sections for every segment is electrically isolated from its service environment. In addition, the active EAP sections within each segment would be electrically isolated from adjacent segments as well.

For each segment of the catheter, there are a total of n active EAP sections. The value of n is in the range of 2 to more than 2. A single active EAP section consists of multiple layers of EAP with a conductive polymer layer or any type of conductor layer deposited on the surface of each layer in the section, which serves as the conductive electrode required for actuation (FIG. 9).

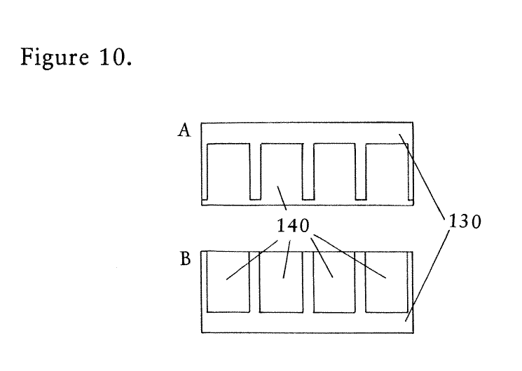

The multiple layers are achieved by electroding two common flat EAP specimens, as seen in FIG. 10, and then laying them atop one another with their electrodes aligned. The electrode spacing along the width and length of the flat EAP specimen is such that as the two specimens are rolled into a tube shape, each overlapping circumference positions the next electrode layer directly on top of the preceding electrode. The result of the electrode layering in this manner is seen in FIG. 9. In this way, the common EAP specimen rolled into a tube serves as the structure for the catheter.

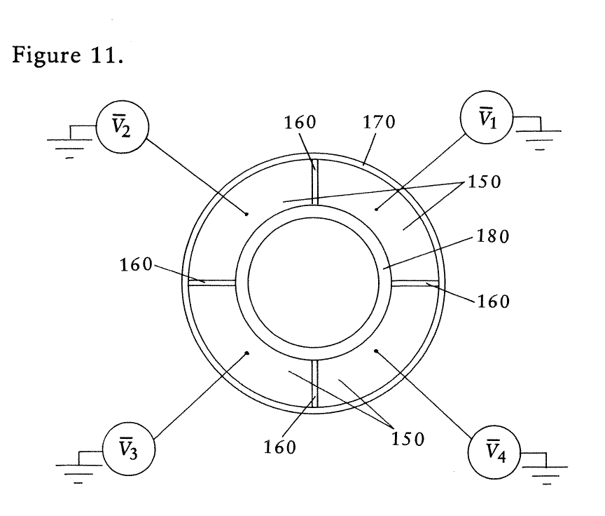

The stretched axis of each EAP layer is aligned with the axis of the catheter tube axis. Every other layer in the active EAP section is connected to the positive polarity and the remainder is connected to the negative polarity of the corresponding driving signal generator (FIG. 9 and FIG. 11). Because a common ground is used for all active EAP sections in all segments, the negative polarity for all active EAP sections in all segments are electrically connected to one another (FIG. 11). When energized by the driving signal generator, the active EAP section extends along its stretched axis. Since the axial extension of the active EAP occurs at some nonzero eccentricity from the neutral axis of the catheter cross section, a nonzero moment is generated. This bending moment causes the catheter tip to deflect relative to its position prior to the active EAP being energized. The desired catheter shape results from the superposition of the deflections achieved by activating various active EAP combinations of segments and sections within the segments using various magnitudes of driving voltage signal.

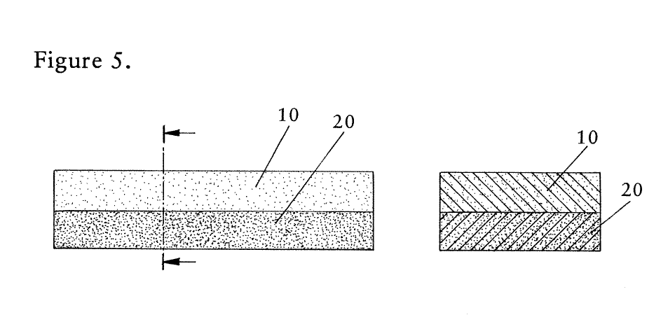

FIG. 5 shows the cross section of a hybrid actuator consisting of a SMP layer 20 bonded to an active (electrode) EAP layer 10. The hybrid actuator can have many different kinds of combination of EAP and SMP. For example, a hybrid actuator can have an EAP layer bonded between two SMP layers, or one EAP layer and one SMP layer bonded together. Electroactive polymer layers are shown on the top of the cross section. The lower portion of the cross section is made from a shape memory polymer. The electroactive polymers are positioned away from the neutral axis to yield a nonzero eccentricity thereby producing a moment responsible for actuator displacement. After an applied electric field deflects the catheter, the shape memory polymer is energized to maintain the shape of the deflected catheter. As the shape memory polymer maintains the shape of the catheter, the electric field can be removed from the electroactive polymer without the structure returning to its initial shape.

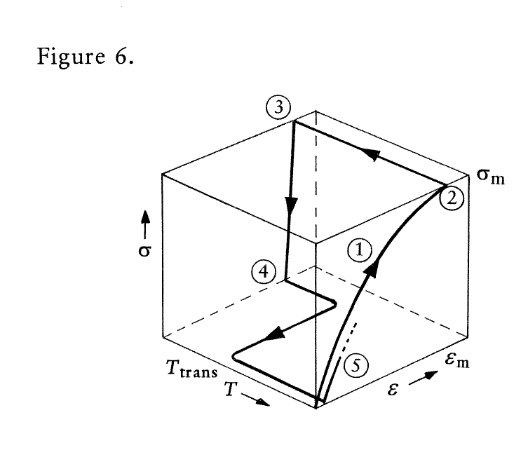

FIG. 6 shows the use of a SMP for the intended application. In the path labeled 1, the SMP temperature is above Tₜᵣₐₙₛ and hence exhibits a very low elastic modulus (<100 MPa) and can be easily strained to a strain value of εₘ under a stress of σₘ. The SMP is then cooled down to a temperature below Tₜᵣₐₙₛ (labeled path 2 to 3) at which point the SMP experiences a memory effect. Even after removing the applied stress σₘ (labeled path 3 to 4), the SMP can still retain the strain value of εₘ.

One advantageous feature of a SMP is the very large elastic modulus change over a very narrow temperature range. Therefore, for an EAP actuator with a SMP layer as illustrated in FIG. 5 at a temperature T>Tₜᵣₐₙₛ of the SMP, at which temperature the SMP has a very low modulus, the SMP can easily be reshaped. Applying an electric field to one EAP layer will cause a bending motion similar to that in FIG. 4. Reducing the temperature to below Tₜᵣₐₙₛ while the EAP is still under the electric field will maintain the articulated position, and meanwhile the SMP will memorize this articulated state. At T<Tₜᵣₐₙₛ, removing the electric field from the EAP film will not affect the articulated position as the memory effect of the SMP will maintain the new shape.

FIG. 7 shows an orthographic view of entire length of the steerable catheter. The total number of active segments along the length direction of the catheter can be from one to more than 4. Within each segment are separate active sections 30 electrically isolated by inactive (non-electroded) EAP material 40. The inner diameter of the active EAP segments is electrically isolated from the inner diameter volume of the EAP tube by an electrical insulator 50. The electrical micro-steerable catheter with four segments along the length and 4 sections 30 around the circumference is illustrated in this figure. It shows the separate electroactive polymer segments positioned around the circumference of the steerable catheter tube. In addition, it shows a plane sliced from the cross section, which is detailed in FIG. 8.

FIG. 8 shows the cross section of steerable catheter of FIG. 7 with 4 active EAP sections 60 around the circumference. The number of active sections can be from 2 to more than 10. This cross sectional view also includes the inner diameter electrical isolator 80 which electrically isolates the active EAP sections from the inner diameter volume of the EAP tube. In addition, the figure also shows the location of the SMP 100 which serves to maintain the deflected shape of the electrical micro-steerable catheter after the electric field responsible for the deflected shape is removed. Also included is the inactive EAP material 70 which serve to electrically isolated each active EAP section 60 within each segment. It shows the position of the shape memory polymer for the hybrid design (SMP may or may not need to be present in an electrical micro-steerable catheter). It shows the separate electroactive polymer sections within each segment, positioned around the steerable catheter circumference. It also shows the electrical insulating material that surrounds the catheter, which isolates the conducting signals from the steerable catheter's service environment. It also shows that each electroactive polymer section in a segment is electrically isolated from one another.

FIG. 9 shows how an electroactive polymer section within a segment is layered and shows the EAP 110 layering with electrodes 120 (a cross sectional cut parallel to the axis of the catheter tube and through a single active EAP section) of an electroactive polymer section and electric pattern within a section of a segment according to an embodiment of the disclosure. The positive and ground electrodes are connected at the two ends as indicated by the grounding symbol and the voltage symbol. It shows how each layer of the electroactive polymer has a layer of conducting polymer or other conductive electric layer deposited on its surface (the figure is not drawn in proportion and in reality, the thickness of the conductive electric layer thickness is very thin, at least 20 times or more thinner than the EAP layer). In addition, it shows how every other layer shares the same polarity, which is connected through the conducting polymer layer or conductive electric layer. It shows that one polarity is connected to ground and the other to an electric voltage.

FIG. 10 shows a portion of two continuous sheets of electroactive polymer 130 with discrete areas of conducting polymer (or other electrode) 140 deposited on their surfaces. This figure shows only one segment along the axis of the tube, however, there can be more than one segment along the axis of the catheter tube. The sheets are laid atop one another with their electrodes facing the same direction and rolled together to form a tube. As the sheets are rolled, the conducting polymer electrodes overlap creating the layered electroactive polymers detailed in FIG. 9. As the sheets are rolled the tube circumference increases, requiring that the conducting polymer electrodes be longer and spaced farther apart to accommodate the increase in arc length. This is done to create the layered electroactive polymer detailed in FIG. 9.

FIG. 11 shows how each electroactive polymer section in a segment is electrically connected to its driving electric field. It shows how each electroactive polymer section 150 in a segment is electrically connected to its separate driving voltage for the electrode pattern in FIG. 9. As detailed earlier, this figure also shows the inactive (non-electroded) EAP material 160 which serves to electrically isolate each active EAP section 150 within each segment. In addition, the electrical isolator 180 separating the inner diameter volume of the EAP tube from the inner diameter of the active EAP sections is shown. Also, the electrical isolator 170 which isolates the entire electrical micro-steerable catheter from its service environment is shown. FIG. 11 also shows that adjacent electroactive polymer sections can share the same electrical ground to limit the number of conductors needed to supply the driving electric fields. In addition, it shows that each electroactive polymer section in each segment has its own driving voltage.

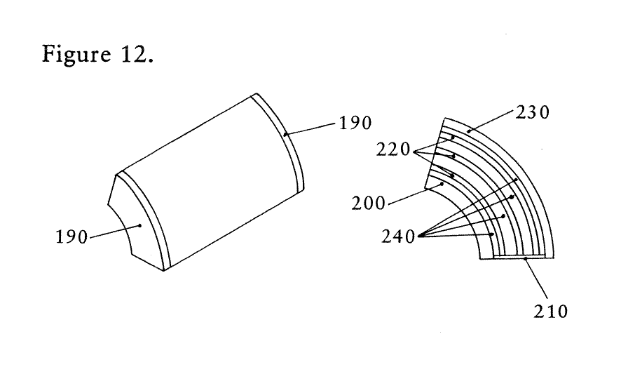

FIG. 12 shows an orthographic view of one electroactive polymer segment from one electroactive polymer section. The electrodes 220 applied each layer of EAP material 240 are detailed in the cross sectional views of the orthographic view. At the ends of the segment are the electrodes 190 that connect every other layered EAP electrode in the section to the driving signal. This figure also shows the inactive EAP material 210 which serves to electrically isolate each active EAP section within each segment. The electrical isolator 200 separating the inner diameter volume of the EAP tube from the inner diameter of the active EAP section is shown. Also, the electrical isolator 230 which isolates the entire electrical micro-steerable catheter from its service environment is shown. It shows the electrical isolation of the electroactive polymer material, which has a conducting polymer electrode, from adjacent electroactive polymer sections. FIG. 12 shows the electrical insulator on the inner and outer diameters of the electroactive polymer section, which electrically isolates the electroactive polymer segment from the inner tube volume and its service environment, respectively.

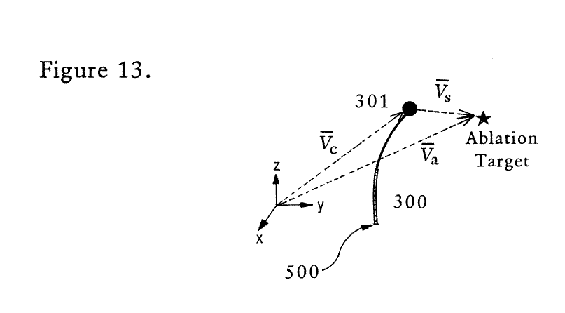

The electrical micro-steerable catheter design lends itself to programmability as the catheter tip position can be precisely controlled by voltage signals applied to the electroactive polymer sheath, which are well suited to computer control. As shown in FIG. 13, control (500), preferable a computer controller, can be made to vary the applied voltages to different segments and sections of the electroactive polymers to induce a desired steerable catheter shape for the catheter tip to reach the target in the body. For instance, the values of the applied voltages necessary to achieve a desired catheter shape for the tip to reach a predetermined target in the patient body can be directly input into the catheter controller. The field value is stored until the desired shape is called upon by the user.

In addition to programmability, there exists the capacity to target specific spatial locations of interest that are within the range of the catheter tip movement (actuation range). The actuation range of the catheter tip comprises of all spatial locations that the catheter tip can reach through only the shape change (bending to different directions and bending degree) of its steerable portion of the catheter. FIG. 13 illustrates the catheter 300 with an ablation transducer tip 310 and an ablation target. FIG. 13 shows the catheter tip located at vector position V̅_{c} and a single ablation target point located at vector position V̅_{α}, with respect to a global coordinate system in three dimensional space. The location vectors are separated by a vector V̅ₛ= V̅_{α}− V̅_{c} with magnitude V=| V̅ₛ|. In targeting with the catheter tip (310), the desire is to position the catheter tip (310) at the ablation target through only the shape change of the steerable catheter (300) by actuating the electroactive polymer sheath. As such, the value of V must be minimized through variations in the applied voltage to each individual actuator section in each actuator segment. User defined convergence to the target is achieved when the catheter tip is within an acceptable tolerance from the ablation target as dictated by the transducer limitations. Written mathematically, the optimization problem of minimizing V can be written using an objective function as:

minimize: V(Ē)+1000×max(0,g)²

where: g=V(Ē)−ε

subject to: E_{Li}≦Eᵢ≦E_{Ui}∀i

where Ē is a one dimensional vector of the applied voltage values, Eᵢ, for each electroactive polymer section in each segment. The inequalities represent the lower and upper bounds on the applied voltage given as E_{Li} and E_{Ui} respectively, where the index, i, is the element index value in the one dimensional vector of applied voltage values, Ē. The equality value g represents the precision of location with respect to the target. The variable ε is a user defined measurement value that dictates the required precision of location of the catheter tip (310) to the target. Using this approach, the distance to a single target point of interest would be minimized using an optimization algorithm. The objective function can be many different types of functions, each one tailored to the specific optimization routine used.

For example, an electrical micro-steerable catheter that uses 1 segment along its length and 4 sections. The lower and upper bounds on the applied voltage are E_{Li}=0 MV/m and E_{Ui}=140 MV/m (voltage=E×EAP layer thickness. For a 3 μm thick film, the voltage is 140×3=420 V) respectively. The value of the upper bound is determined by the electroactive polymer. The precision value ε=1 mm is used in this example (the precision value can be less than 1 mm such as 0.1 mm). Simulated Annealing is employed as the optimization algorithm as it is well suited for optimizing constrained nonlinear functions, and helps to ensure that a global minimum within the design space is reached. Convergence is determined by a user defined acceptable targeting precision value. A precise distance from the catheter tip to the target is defined as less than 1 mm.

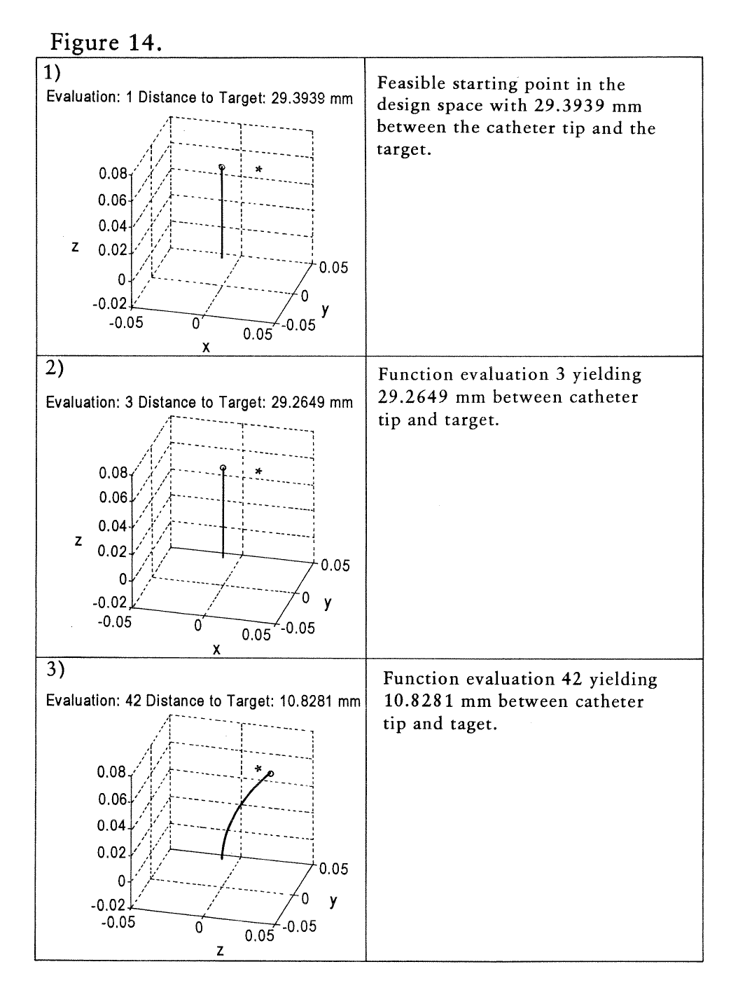

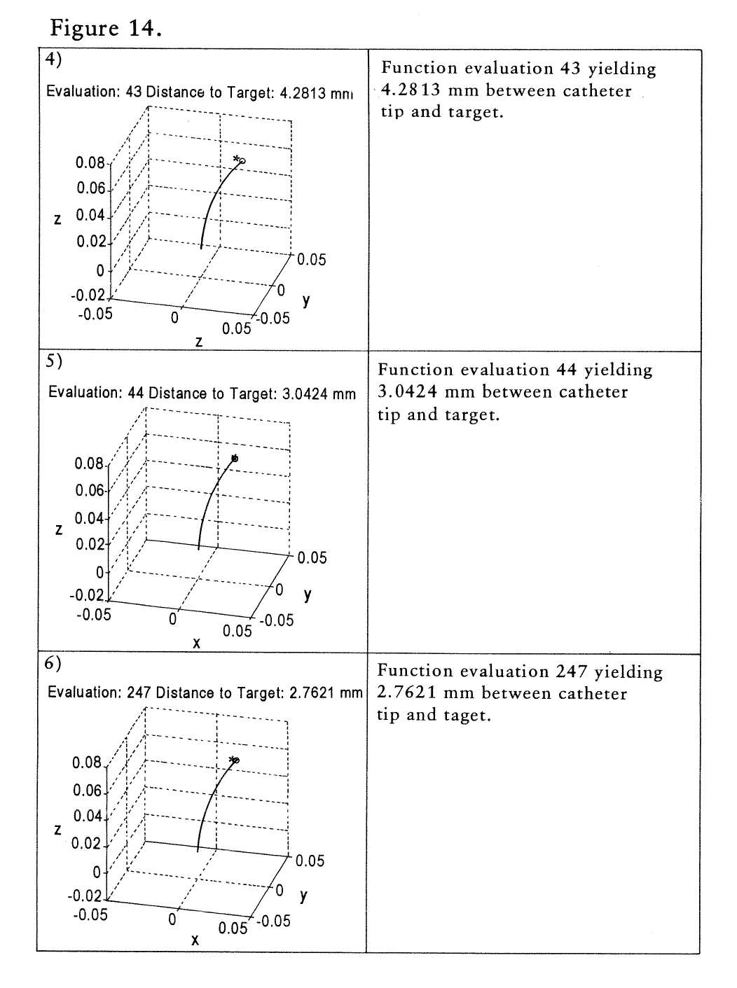

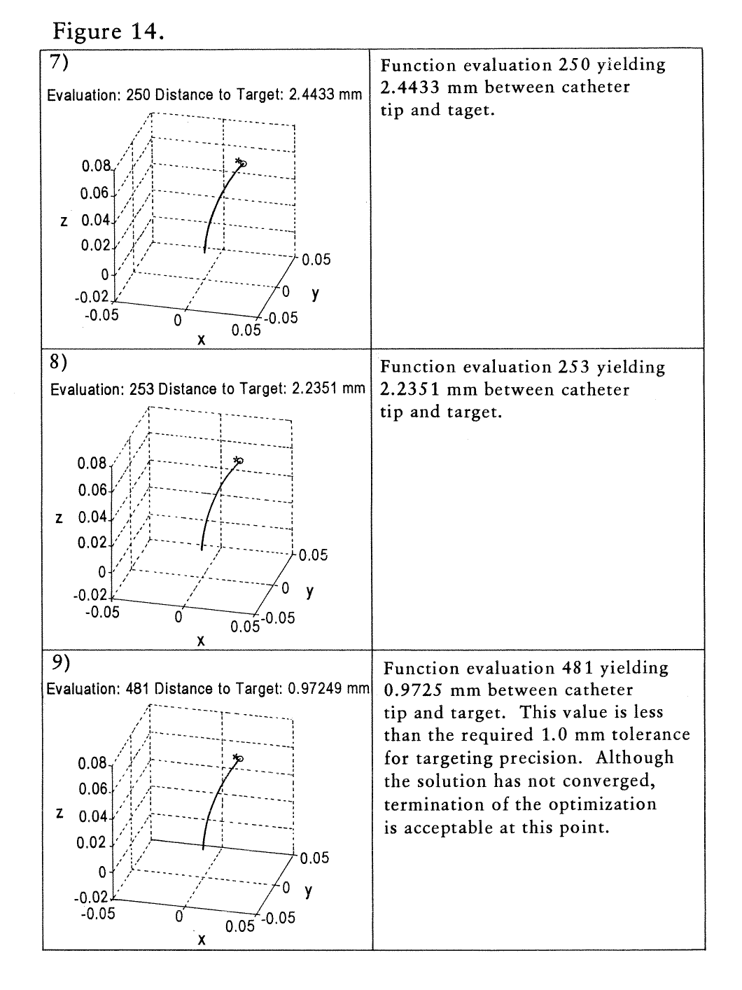

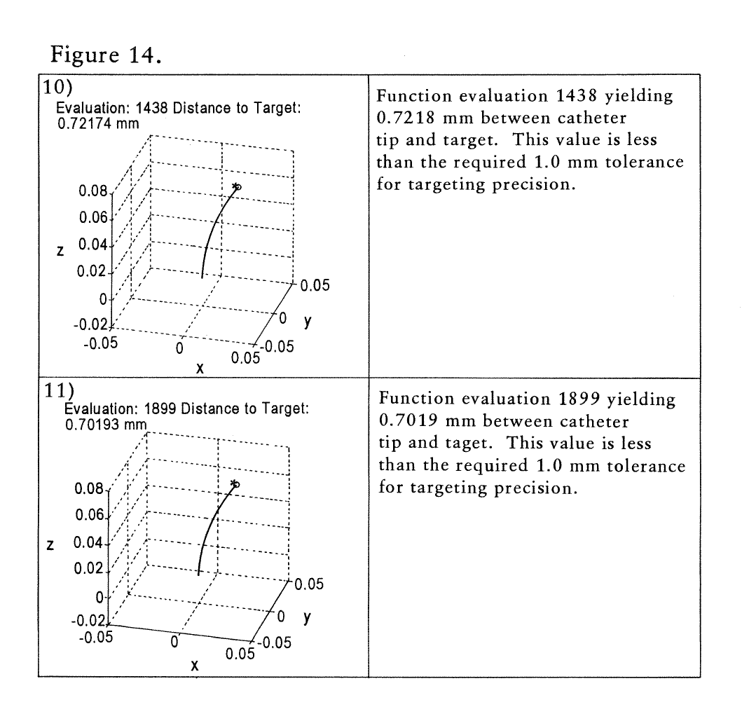

FIG. 14 gives the results from the optimization showing both the resulting iterated shape and the remaining distance from the catheter tip to the target point. The total computational run time for the optimization to converge to the final solution for this case is 14.51 seconds (using an Intel® Core™ dual CPU 4300 at 1.8 GHz each). This run time value can be less if the convergence criteria concerning the distance from the catheter tip to the target of less than 1 mm is increased or a faster computer processor is employed. Also, the convergence time can be reduced if, at the first occurrence of a feasible solution yielding a catheter tip to target distance of less than 1 mm is encountered, the optimization is terminated as opposed to allowing it to continue to an even closer value. This is illustrated in shape 9 through shape 11 in FIG. 14. The distance between the catheter tip and the target is less than the required 1 mm for shape 9, however, the optimization routine did not reach convergence as defined computationally, although it did reach convergence based on the user definition. Hence, two more solutions were developed that are within the 1 mm tolerance and are even closer to the target. As such, the optimization routine could be terminated upon reaching shape 9 without continuing the evaluation to shape 11.

In an another example, the same electrical micro-steerable catheter that uses 20 segments along its length and 4 sections in each segment is used. Such a configuration allows for more complex shapes than the first case, and as a result offers a larger actuation range. The lower and upper bounds on the applied voltage are E_{Li}=0 MV/m and E_{Ui}=140 MV/m respectively. The precision value ε=1 mm is used in this case, as for the previous. Simulated Annealing is again employed as the optimization algorithm as it is well suited for optimizing constrained nonlinear functions, and helps to ensure that a global minimum within the design space is reached. Convergence is determined by a user defined acceptable targeting precision value. For this case, a precise distance from the catheter tip to the target is defined as less than 1 mm.

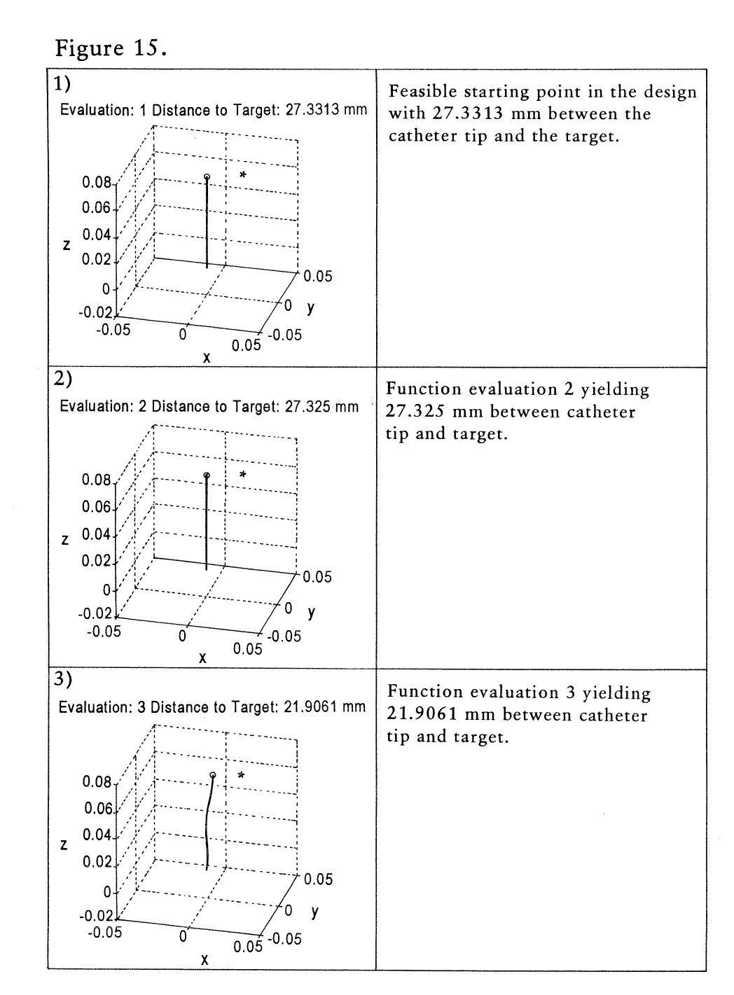

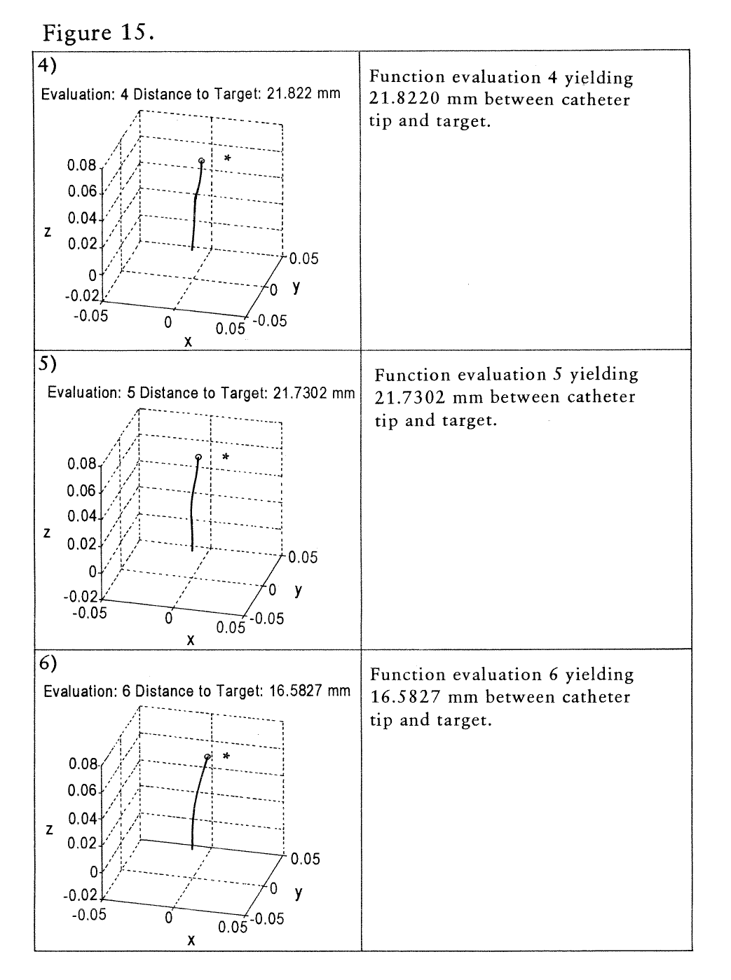

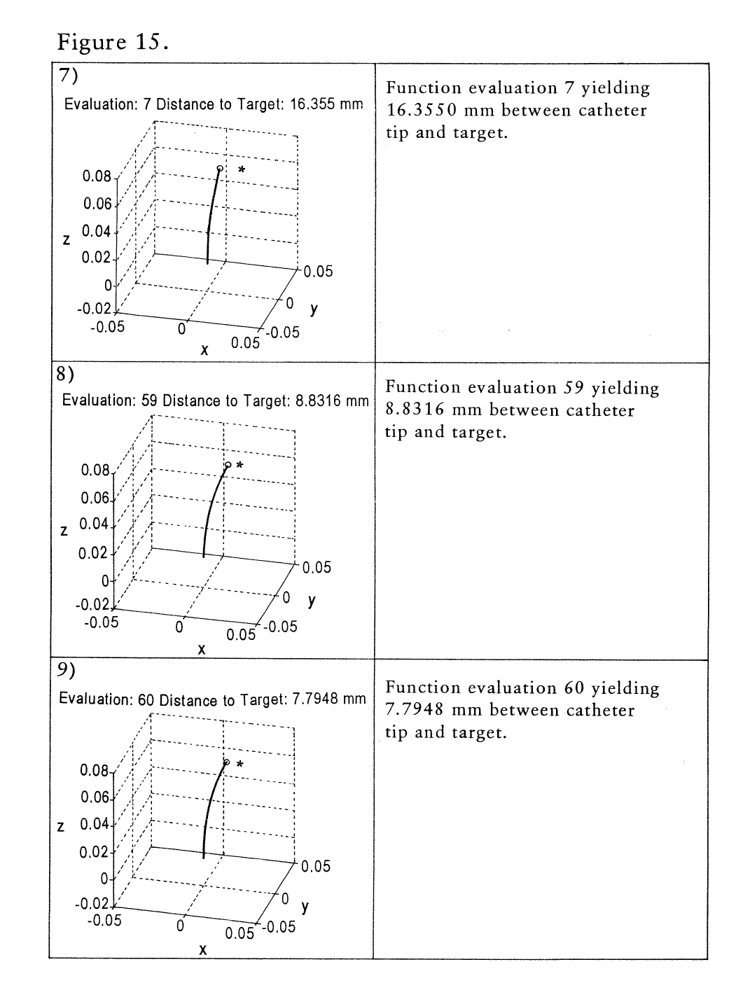

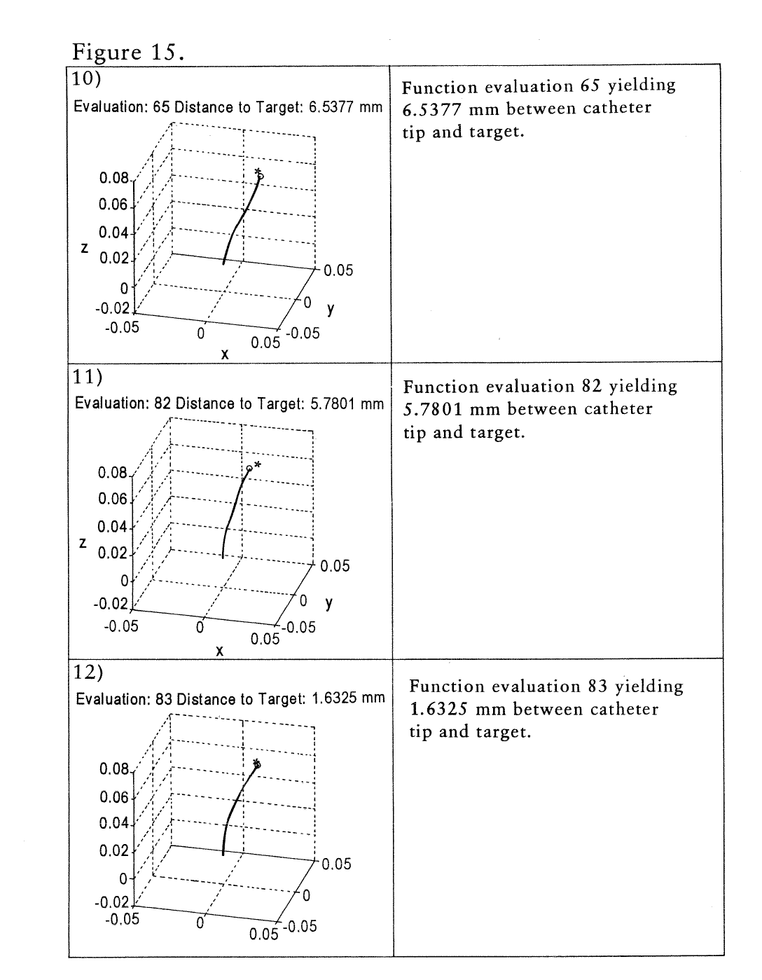

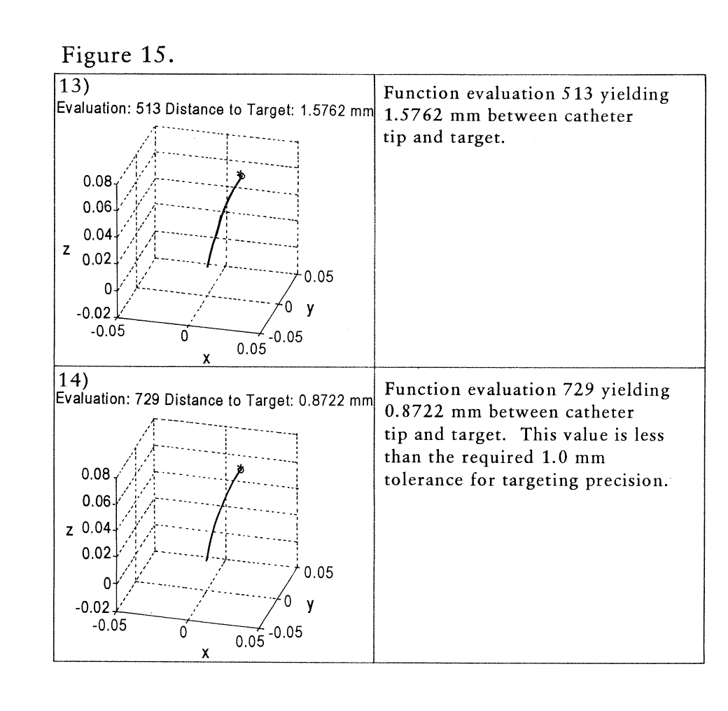

FIG. 15 gives the results from the optimization showing both the resulting iterated shape and the remaining distance from the catheter tip to the target point. The total computational run time for the optimization to converge to the final solution for this case is 141 seconds or a little more than 2 minutes (using an Intel® Core™ dual CPU 4300 at 1.8 GHz each). As indicated before, this run time value can be less if the convergence criteria concerning the distance from the catheter tip to the target of less than 1 mm is increased or a faster PC is employed. Also, the convergence time can be reduced if, at the first occurrence of a feasible solution yielding a catheter tip to target distance of less than 1 mm is encountered, the optimization is terminated as opposed to allowing it to continue to an even closer value. However, this characteristic of computationally defined convergence was not encountered in this case study as it was in the first case study.

If a continuous line of ablations must be performed, then the line must be discretized into closely spaced discrete points, each serving as a single point ablation target. The distance between target points is determined by the ablation transducer resolution necessary to affect a continuous line of ablations.

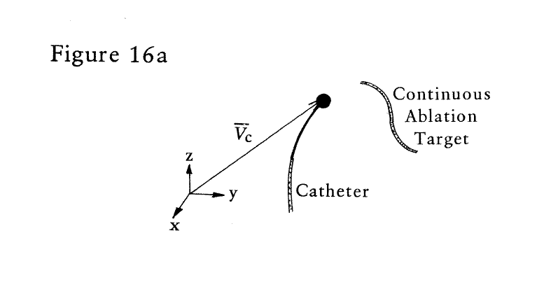

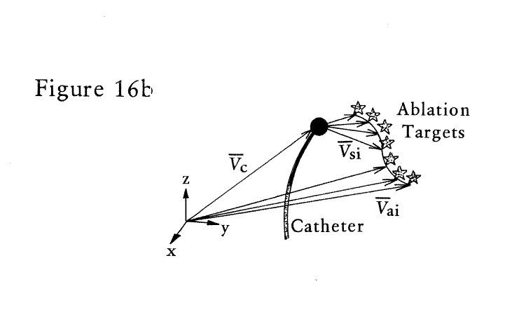

FIGS. 16a and 16b demonstrate discretization. The optimization is run for each point, located at V̅_{αi}, where the previous target point can serve as the feasible starting point for the following target point optimization run. Determining the feasible starting points in this manner reduces the computational time required to perform the optimization as the starting point is very close to the desire solution in the design space. In this way optimization values are achieved and the respective voltage value that achieves that shape is stored on a computer. After the optimization determines all the necessary voltage values, each voltage value can be played back in sequence to track the ablation line.

It is important to note that for either the single or multiple targeting routines, all the optimal electric field values are determined in a virtual environment, as on a computer. As each field for each target of interest is determined, those values are stored for play back at the completion of the virtual targeting routine.

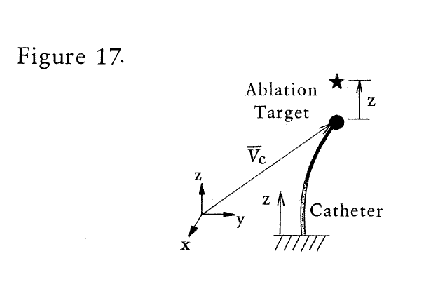

An additional spatial degree of freedom that may or may not be declared as a design variable in the optimization is the translational displacement, z, of the catheter base, as shown in FIG. 17. This displacement is a result of the user either pushing or pulling the catheter into or out of the body. The value of this degree of freedom is that it is possible that a target point may be out of the actuation range of the catheter tip. As such, by including the translational displacement of the base of the electroactive polymer portion of the catheter, these points may then be reached. If an optimization evaluation of a target point shows that it cannot reach the target point (as illustrated in FIG. 13), then this translational displacement degree of freedom can be added to help ensure that the computational evaluation produces a converged solution that is within the user defined precision.

It will be understood that certain of the above-described structures, functions and operations of the above-described preferred embodiments are not necessary to practice the present disclosure and are included in the description simply for completeness of an exemplary embodiment or embodiments. In addition, it will be understood that specifically described structures, functions, and operations set forth in the above-referenced patents can be practiced in conjunction with the present disclosure, but they are not essential to its practice. It is therefore to be understood, that within the scope of the appended claims, the embodiments of the disclosure may be practiced otherwise than as specifically described without actually departing from the spirit and scope of the present disclosure.

1-23. (canceled)

24. An electrically actuated catheter comprising an electroactive polymer (EAP) for steering the catheter, wherein the EAP is selected from the group consisting of polyvinylidene fluoride (PVDF) based ter-polymers and irradiated PVDF based polymers.

25. The catheter of claim 24, wherein the EAP is selected from the group consisting of P(VDFₓ-TrFE_{y}-CFE_{1-x-y}), P(VDFₓ-TrFE_{y}-CTFE_{1-x-y}), Poly(VDFₓ-TrFE_{y}-vinylidene chloride_{1-x-y}), poly(vinylidene fluoride-tetrafluoroethylene-chlorotrifluoroethylene), and wherein x is in the range from 0.5 to 0.75, and y is in the range 0.45 to 0.2 and x+y is less than 1.

26. The catheter of claim 24, wherein the EAP is selected from the group consisting of:
high energy irradiated PVDF based polymers, wherein the high energy irradiation includes electron, γ-ray, and/or α-ray, and
wherein the PVDF based polymer can be selected from P(VDFₓ-TrFE₁₋ₓ), P(VDPₓ-CTFE₁₋ₓ), P(VDFₓ-CFE₁₋ₓ), P(VDFₓ-HFP₁₋ₓ) (HFP: hexafluoropropylene), where x is in the range from 0.5 to 0.95.

27. The catheter of claim 24, wherein the EAP has an elastic modulus of greater than 0.5 GPa.

28. The catheter of claim 24, wherein the EAP is selected from the group consisting of:
P(VDFₓ-TrFE_{y}-CFE_{1-x-y}), P(VDFₓ-TrFE_{y}-CTFE_{1-x-y}), Poly(VDFₓ-TrFE_{y}-vinylidene chloride_{1-x-y}), poly(vinylidene fluoride-tetrafluoroethylene-chlorotrifluoroethylene), poly(vinylidene fluoride-trifluoroethylene-hexafluoropropylene), poly(vinylidene fluoride-tetrafluoroethylene-hexafluoropropylene), poly(vinylidene fluoride-trifluoroethylene-tetrafluoroethylene), poly(vinylidene fluoride-tri fluoroethylene-vinyl fluoride), poly(vinylidene fluoride-tetrafluoroethylene-vinyl fluoride), poly(vinylidene fluoride-trifluoroethylene-perfluoro(methyl vinyl ether)), poly(vinylidene fluoride-tetrafluoroethylene-perfluoro (methyl vinyl ether)), poly(vinylidene fluoride-trifluoroethylene-bromotrifluoroethylene, polyvinylidene), poly(vinylidene fluoride-tetrafluoroethylene-chlorofluoroethylene), poly(vinylidene fluoride-trifluoroethylene-vinylidene chloride), and poly(vinylidene fluoride-tetrafluoroethylene-vinylidene chloride);
and wherein x is in the range from 0.5 to 0.75, and y is in the range 0.45 to 0.2 and x+y is less than 1.

29. The catheter of claim 24, wherein the EAP is in the form of a sheath on the catheter.

30. The catheter of claim 29, further comprising a distal tip, wherein the EAP sheath is about 5 to about 10 cm in length from the distal tip.

31. The catheter of claim 29, wherein the EAP sheath is electrically divided into two or more sections around the circumference of the catheter.

32. The catheter of claim 29, wherein the EAP sheath is electroded into one or more segments along the lengthwise direction of the catheter.

33. The catheter of claim 32, wherein the total number of the segments is any number larger than one.

34. The catheter of claim 33, wherein each segment is electrically isolated from another segment so that each segment is individually actuated.

35. The catheter of claim 24, wherein the catheter comprises multiple (EAP) layers rolled into a sheath.

36. The catheter of claim 35, wherein the multiple EAP layers are uniaxially stretched films.

37. The catheter of claim 35, wherein the multiple EAP layers are in non-stretched form.

38. The catheter of claim 35, wherein the sheath comprises a shape memory polymer layer.

39. The catheter of claim 35 wherein the sheath comprises an additional shape memory polymer (SMP) layer, and
wherein the SMP layer has a glass transition temperature between 38 to 45° C.

40. The catheter of claim 24, wherein the catheter has a distal tip position that can be moved from a range of less than one millimeter to several centimeters by energizing the EAP.

41. The catheter of claim 24, wherein the steerable portion of the catheter is comprised entirely of EAP.

42. A programmable catheter system, comprising the electrically actuated catheter of claim 24.
